(19) United States
(12) Patent Application Publication
(10) Pub. No.: US 2025/0332454 A1
(43) Pub. Date: Oct. 30, 2025

(54) MEASURING AND TRACKING TRANSCRANIAL FOCUSED ULTRASOUND STIMULATION AND MODULATION EFFICACY

(71) Applicant: Sanmai Technologies, PBC, Sunnyvale, CA (US)

(72) Inventors: Tomokazu SATO, Minneapolis, MN (US); Christopher DAFT, Tucson, AZ (US); Timothy MULLEN, Hidden Meadows, CA (US)

(21) Appl. No.: 19/098,837

(22) Filed: Apr. 2, 2025

Related U.S. Application Data
(60) Provisional application No. 63/640,591, filed on Apr. 30, 2024.

(51) Int. Cl. A61N 7/00

(52) U.S. Cl. CPC A61N 7/00; A61N 2007/0026

(57) ABSTRACT

tFUS delivery and tracking systems and methods assess target location accuracy and therapy efficacy by measuring the specific and predicted downstream effects in individualistic responses to tFUS waveforms. The downstream effects include physiological, stress, mood, movement, attention measurements, subjective reports, task-based performance, etc. The measurements are performed before, during, and between tFUS sessions intermixed with optional control periods or sessions. In an embodiment, when a target brain region does not offer any immediate readouts but is surrounded by regions that may, these latter regions can be used instead for triangulation or waveform optimization. Individual and group tracking methods help identify useful measurement modalities and the expected direction and magnitude of change in response to therapy. A method that enables individualized functional targeting in a non-clinical setting by optimizing cost and complexity is described.

### CROSS-REFERENCE TO RELATED APPLICATION

This application claims the priority benefit of U.S. Provisional Application No. 63/640,591, filed Apr. 30, 2024 and entitled “MEASURING AND TRACKING TRANSCRANIAL FOCUSED ULTRASOUND STIMULATION AND MODULATION EFFICACY”, the contents of which are incorporated herein by reference in its entirety.

### TECHNICAL FIELD

This application deals with transcranial-focused ultrasound efficacy, specifically measuring the efficacy of tFUS and tracking it across a population of subjects.

### BACKGROUND

Transcranial-focused ultrasound systems can be used to help treat several types of mental illnesses. tFUS can target deep brain structures from which direct physiological measurements cannot be made without using fMRI or other resource-intensive methods. However, human variability in head dimensions and specific brain region localization can make targeting specific brain regions hard. Even with perfect structural-level anatomical information, such as CT (computerized tomography) scans for bone density and MRI (magnetic resonance imaging) for brain shape, there may not be sufficient data for targeting. These “spatial targeting” methods rely solely on the assumption that each individual's brain and brain regions are arranged identically with just some linear stretching to match the individual's head shape to a generic template.

FMRI (functional magnetic resonance imaging) with tasks that use specific brain regions of interest to target (or brain regions that can be used reliably as nearby anatomical markers) could be helpful. However, due to the cost and accessibility of fMRI, such methods are not adoptable if the goal is the widescale use of tFUS as a therapeutic modality. FMRI tasks take far longer than a simple anatomical image with more complex methods necessary such as sensory stimuli or tasks in a magnetic environment. Thus, there is still a need to have high precision, individualized targeting of brain regions assisted by modalities that can be more readily and practically implemented and used by a larger subject population. There is a need for “functional targeting,” which uses specific and predicted measurements of downstream effects of neuromodulation to optimize individual targeting and therapy parameters.

### BRIEF DESCRIPTION OF THE DRAWINGS

FIG. 1 shows tFUS delivery and tracking system 100 with measurement systems to track the efficacy of the tFUS waveforms.

FIGS. 2A-2D show sample measurements performed by System 100 to track the effects of tFUS stimulation and modulation.

FIGS. 3A and 3B illustrate targeting examples using System 100. FIG. 3A shows an example of direct targeting, while FIG. 3B shows indirect targeting.

FIG. 4 shows an exemplary method 400 used by System 100 for indirect targeting.

FIG. 5 shows an exemplary method 500 performed using System 100 to evaluate and improve the efficacy of tFUS stimulation and modulation.

FIG. 6 shows an exemplary measurement 600 performed using System 100, illustrating individual versus group measurement.

FIG. 7 is an exemplary method 700 performed using System 100 to adjust individual and group measurements.

FIG. 8 shows an exemplary Method 800 that enables functional targeting of a subject for at-home use.

### DETAILED DESCRIPTION

tFUS delivery and tracking systems and methods assess target location accuracy and therapy efficacy by measuring the specific and predicted downstream effects in individualistic responses to FUS waveforms. The downstream effects include physiological, stress, mood, movement, attention measurements, subjective reports, task-based performance, etc. The measurements are performed before, during, and between tFUS sessions intermixed with control periods. In an embodiment, when a target brain region does not offer any immediate readouts but is surrounded by regions that may, these latter regions can be used instead for triangulation or waveform optimization. Individual and group tracking methods help identify useful measurement modalities and the expected direction and magnitude of change in response to therapy. Note that for using such measures for “functional targeting”, one may use a tFUS waveform that is different from that used in therapy, and possibly with different or alternative effects. For example, one may want to reduce activity in the amygdala for a patient with anxiety disorder or PTSD and use a “depressing” waveform. However, for the sake of “functional targeting”, one may use a stimulating (i.e. “activating” or “excitatory”) tFUS waveform to elicit strong and measurable effects. A method is described that enables individualized functional targeting in a non-clinical setting by optimizing cost and complexity.

tFUS denotes a transcranially delivered “focused” ultrasonic waveform in time and space that elicits targeted activity changes in the brain region to which it is “focused.” Note that “focused” may be a spatial focus or denote focally induced brain activity changes. In contrast, the spatial aspects of the ultrasound field itself may not fall under the standard definition of “focused.” “Modulating tFUS waveform” or “tFUS waveform” or “tFUS” is a tFUS waveform that modulates a brain area. This could be excitatory, inhibitory, or modulate the magnitude of induced activity in the brain region in response to other stimuli, including incoming neural inputs to the brain region, tasks, sensory stimuli, and other tFUS waveforms or affect the local neural oscillation timing, phase, frequency, amplitude, or shape.

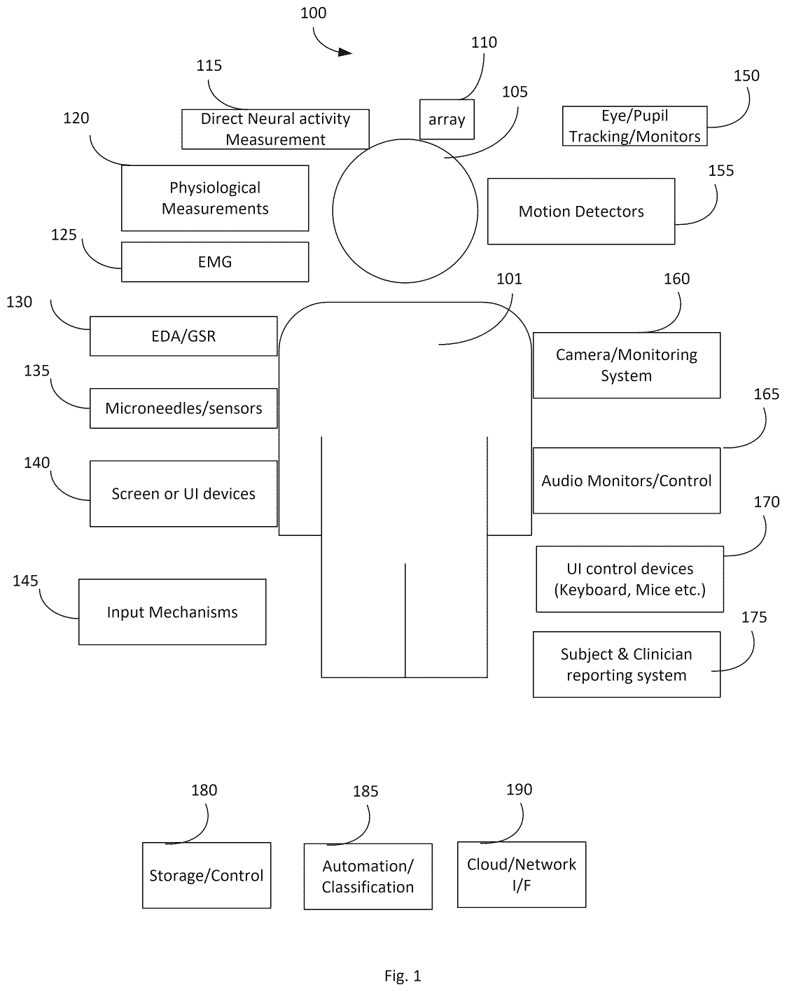

FIG. 1 shows tFUS delivery and tracking system 100 with measurement systems to track the efficacy of the tFUS waveforms. FIG. 1 shows subject 101 with a transducer array assembly 110 attached to their head 105. Transducer array assembly 110 optionally includes anatomical imaging (targeting) systems (not shown in the figure) such as ultrasound-based, CT and MRI scans, LIDAR, and cameras for skull shape assessment and neuronavigation. Alternatively, images obtained from such systems beforehand can be used and aligned with the subject using facial features, etc. They aid with the guidance of the tFUS waveforms. Imaging or spatial targeting systems that focus purely on the static spatial aspects of the head and brain can also offer initial feedback on the accuracy of tFUS targeting. However, there can be limitations without more detailed individualized functional anatomical information.

tFUS delivery and tracking System 100 includes direct neural activity measurement system 115 for directly measuring neural activity. Examples of such systems include EEG (Electroencephalography), MEG (magnetoencephalography), fMRI, fNIRS (functional near-infrared spectroscopy), etc. Direct neural activity measurement systems 115 can be considered “neurophysiological targeting.” For example, EEG is a measure of the brain's electrical activity and often cannot reveal the specific brain region that was modulated. To explain, one could be attempting to target the primary visual cortex (V1, which, for example, when stimulated by transcranial magnetic stimulation, leads to phosphenes, or percepts of light without any actual change in light entering the eyes) by looking for an evoked potential seen in EEG using a stimulatory tFUS waveform. However, suppose there are no significant differences in responsivity to tFUS or resulting evoked potential shapes in EEG, especially in a related cortical area. In that case, one could easily be targeting a higher-level visual cortex (such as V5, which, when stimulated by transcranial magnetic stimulation, leads to alteration of motion percept) or some other cortical area with similar evoked potential shapes expected from the tFUS waveform. Thus, one would not differentiate whether the targeting was accurate. While EEG-based targeting could sometimes be used for anatomical targeting, primarily based on expected connectivity-based changes, these measured changes are still localized and originate from neural activity changes.

EEG can still, in some cases, yield additional real-time insight. Conditions such as anxiety and depression are suggested to involve changes in connectivity between regions such as the amygdala and various locations of the cortex or regions of the cingulate cortices (including the posterior) to the lateral orbitofrontal cortex. For example, high-density EEG or fNIRS recordings from the frontal portions alone could be more practical, especially as they obliviate the need to record through hair. The strengths of oscillations (alpha, delta, and theta) are often found in the frontal cortices and indicate the mental state or strength of coupling from other regions. Studies in epileptic patients have also demonstrated measurable evoked potentials (albeit using cortical surface or depth electrodes) in cortical regions that could be measured with EEG in response to electrical stimulation of the amygdala or cingulate cortical regions. In addition, some brain regions, like the PCC (posterior cingulate cortex), have direct, strong, and low-latency connections between the left and right hemispheres, such that signatures in EEG may be observable. Finally, oscillation strength during tasks or in response to stimuli can also be used as additional related measures. Thus, EEG still can be a powerful tool for targeting; however, in some cases, it will not be sufficient.

Embodiments of the disclosure focus on “functional targeting,” where specific and predicted measurements of downstream effects of the tFUS waveforms, when properly targeted to intended brain regions within an individual subject, are used to optimize individual targeting and waveform (or therapy) parameters. To highlight this, refer back to the V1 vs V5 example. With only EEG, one may see some oscillatory pulse-like signal in response to tFUS, which may not look very different between the two regions. This could be due to the waveforms representing some form of cortical neural activation with a relatively generic time course and EEG waveform. However, “functional targeting” could use a downstream effect, such as reported effects from the subject, such as did it causes a phosphene (an illusory light flash that one would expect with proper targeting of V1) or a disruption in motion perception (if one were accidentally activating V5). The downstream effects that could be used include physiological measurements 120 such as heart rate, pupil diameter, blood flow changes, breathing, blood pressure monitors, etc. The downstream effects can also be measured based on subjective information, tasks, etc. Subject 101, during treatment or between sessions, reports on their mood, positive or negative feelings, dreams, sleep quality, etc. Subject 101 may also undergo tasks with or without explicit instructions. Non-explicit tasks may present stimuli, audio, objects, or pictures passively (i.e., without actively telling the subject of the relevance) and measure reactions. Explicit tasks beyond subjective reporting may include objective strategic games (risk, memory, etc.) or postural change. Each subject may show differences in which measurement method shows proper targeting, optimized waveforms, etc. Thus, the diversity of available methods is useful. These “functional targeting” methods would likely be used after using “spatial targeting” methods to refine further targeting for an individual subject, especially in early sessions. However, after some adjustments, the proper targeting would be known, and one may not need to do “spatial targeting” at the beginning of the session. The same “spatial targeting” methods (mainly cameras) can be used to record the adjustments and placements during and after the “functional targeting” is done to record the better positioning and aiming of the transducers and used more directly in later sessions. In other words, after one or more sessions, after having obtained the “functional targeting” coordinates and orientations, one may need only simple methods, similar to neuronavigation used normally for “spatial targeting” (fiducial markers, etc.) These may need to be adjusted if there is drift, but not necessarily frequently. These “functional targeting” methods may be used after, before, in conjunction with, or as potential replacements for other more direct neural activity reporting systems 115 (EEG, MRI, fMRI, fNIRS.) In addition, the same camera could be used for measuring facial expression, agitation/movement, etc., and thus could be used in aspects of “functional targeting” and not just “spatial targeting.” These “functional targeting” methods can also rely on subjective reporting and measurements. Thus, these methods, equipment, modalities, etc. are not entirely exclusive; they are different conceptualizations that overlap. The efficacy of tFUS waveforms is evaluated using multiple test waveforms, and subject 101's responses are measured.

### Physiological Measurements

Physiological measurements 120 in System 100 measure readouts of bodily functions, processes, and status such as the circulation and respiration of subject 101 in response to tFUS waveform stimulation or modulation. These include blood pressure, blood flow (in specific arteries, for instance, using Doppler US), blood oxygen saturation, blood or end-tidal CO2 (carbon dioxide), heartbeat strength, heart rate and rate variability, heartbeat electrical waveform (EKG), respiration rate and depth, tidal volume, nasal airflow speed and pressure, oral or nasal thermal measurements, pulse rate, temperature, blood sugar, etc. Physiological measurement 120 measures the timing, amplitude, shape, variability, and relative phases between measurements. Physiological measurement 120 measures the changes due to FUS waveforms. Physiological measurement 120 measures the changes in response to tasks or postural changes. Physiological measurement 120 measures the time it takes for measurements to return to baseline after a change or perturbation in response to a tFUS waveform or a task. Note that a few other measurements that could also be considered physiological measurements are explicitly described separately (EMG, stress measurements) from these more common measurements which are commonly used in the clinic or with wearables.

### EMG

EMG (Electromyography) 125 in System 100 measures facial muscle for facial expressions, induced motion (if the motor area is stimulated), general agitation, force output, etc. Thus, while EMG is a physiological measurement, it can be used as a correlate of other measures indirectly (lots of fidgeting as a sign of nervousness, etc.)

### Stress Measurement

EDA (electrodermal activity) or GSR (galvanic skin resistance) 130 and Microneedles (similar to continuous glucose monitors) or skin surface patch sensors with electrochemical sensors 135 can be used to measure subject 101's stress and sympathetic response to stimulation or modulation by tFUS waveforms or tasks. For example, sweating (as an indicator of stress, anxiety, or nervousness) can be ascertained using EDA 130 or cortisol, adrenaline, blood sugar, etc., indicating stress can be measured by microneedles/electrochemical sensors 135. Timing, amplitude, variability, relative phases between measurements, and time to return to baseline can be measured. In an embodiment, the stress measurement using EDA 130 or microneedles/sensors 135 monitors subject 101's stress response continuously (i.e., 24/7). While the previous methods may be considered physiological, other methods to indirectly measure stress or anxiety can also be used. These could be videographic analysis of facial expressions, amount of fidgeting, etc.

### Mood and Attention Measurement

System 100 measures correlates of attention, exploratory activity, impulses to move or states of agitation, eye movement, and facial movement/expressions. These can be measured using Eye/Pupil tracking/monitors 150, motion sensors/detectors 155, camera/monitoring system 160, audio monitors/control 165, etc. Camera/monitoring system 155 can include head-mounted (similar VR or AR headsets, glasses, etc.), heads-up cameras, smart glasses, other external camera systems, EMG 125, etc. These systems can track and detect head motion, eye movement, facial expression, etc. Audio Monitors/Control 165 measures various sounds. For example, it can measure the subject's voice to detect stress. Audio Monitor 165 can measure breathing sounds to detect the breath rate and depth and corresponding stress level. An AI-based automation/classification system 185 assists in automating the measurement of correlates and classification. These systems are used to quantify a subject 101's state. Correlates of attention and exploratory activity of the external environment, such as pupil size, large eye-directed motions such as gaze, rapid eye movements such as saccades, and exploratory eye and head movements, are measured. Constant eye movements can indicate anxiety, PTSD, or other mood disorders. For instance, PCC (posterior cingulate cortex) may show more biases in the location or direction of eye movement due to its involvement in processing spatial information in memory. In contrast, the amygdala may show more exploratory or anxious eye movement. Correlates measured include facial muscle measurements via EMG 125 and videographic analysis of the subject's facial expression. For example, humans can typically easily rate the facial expressions of others, and in fact, such images are often used in tasks to study the human brain. However, such faces are also identifiable (in categories like happy, sad, angry, and neutral) and even quantifiable using facial landmarks using more standard analysis methods like principal components analysis or more modern techniques like neural nets/artificial intelligence in automation/classification system 185. Certain regions, like the amygdala, are more involved in emotions and emotional processing and are likely to show these effects.

### UI, Input Mechanisms, and System Control

System 100 includes multiple means for subject 101 (and the clinician) to interact with it, including a screen or UI devices 140, Input Mechanisms 145, Audio Control 165, and UI control devices 170. Screen or UI devices 140 include video monitors, touch screens, tablets, VR/AR headsets, smart glasses, etc., allowing subject 101 to receive instructions from System 100 and provide feedback or instructions to System 100. Audio control 165 allows subject 101 to interact with System 100 using audio instead of a visual medium. Audio control 165 could include speakers, headphones, microphones, etc., allowing subject 101 to receive audio instructions, stimuli, or information from System 100. Using Audio Control 165, subject 101 uses voice to provide instructions or feedback to System 100. UI Control Devices 170 and Input Mechanisms 145 allow subject 101 to provide other means for providing feedback or inputs to System 100. They may include a keyboard, mice, or other mechanical or electronic devices. The input mechanism allows subject 101 to provide feedback to System 100. The feedback may be part of subjective reports or in response to tasks. Input Mechanism 145 may include one or more mechanical or electronic buttons (levers, gadgets, or similar structures,) a joystick, a game controller, etc. Input Mechanism 145 can be pressure sensitive (akin to a squeeze or pressure ball) in one embodiment. Such a pressure-based input could be used to gauge the patient's engagement or emotional responses or state if timing and amplitude (strength of squeeze) are used. The buttons can also be used as part of a task, where the subject is asked to rate pictures, etc., or for the subject to provide feedback on the tFUS stimulation or modulation. For example, Input Mechanism 145 includes two buttons: one indicates a positive response to the stimulation or modulation, and the other a negative one. Alternatively, two buttons can indicate up vs down, left vs right, or first vs second (in timing or sequence).

System 100 includes storage/control 180 for the overall control of the System and for storing various results. Storage includes volatile and non-volatile memory, disks, etc. System 100 also includes various network and cloud interfaces 190. The network/cloud interface 190 communicates individual results to a cloud server. It is also used for gathering group data, weighting different measurements, prioritization, targets, waveforms, etc.

Automation/classification 185 includes AI (artificial intelligence) components to aid with classifying and quantifying measurements. Automation/Classification 185 may be distributed where some or all of the processing is performed on the cloud.

### Subjective Reports as Measurements

Subjective reports such as mood, what subject 101 was thinking of, quality of sleep, the occurrence of nightmares, and other subjective reports like sensations may be used as additional tools to help precisely target the tFUS therapy to the correct brain regions such as the amygdala. Things like out-of-body experiences, lucid dreaming, daydreaming, mind-wandering, future imagination, or personal episodic memories may instead reflect more the PCC. In addition, thoughts on the present vs. the past vs. the future, realistic vs. unrealistic, here vs. elsewhere, and self vs. others can reflect what brain regions are targeted.

System 100 provides a reporting system 175 for subject 101 to submit their subjective reports. The reporting system 175, in conjunction with AI-assisted automation/classification 185, can also classify and quantify subjective reports. Subject 101 uses system 175 to report subjective information as part of a task or to report things they experienced during treatment or between sessions (positive or negative feelings and dreams, sleep quality, etc.) Reporting System 175 may include appropriate UI containing forms, questionnaires, etc., allowing Subject 101 to submit reports. Reporting System 175 may provide the UI using a webpage, app, etc. Reporting System 175 is also used by the clinician to report the clinician's observations, reports, etc., of subject 101.

### Direct Use of Measurements (Without Explicit Task)

One can use these various measurements directly. Below are some examples that can be used to check system positioning and coupling.

Using eye/pupil tracking 150 or camera 160, measure gaze direction, pupil size, and other visual cortex parameters when the visual cortex is stimulated to evoke phosphenes.

The ability to target subregions of the visual field can further validate the skull correction algorithms used.

Targeting areas related to emotional valence, such as amygdala parts, can evoke responses. Beyond subjective reports, one can use GSR 130 or microneedles 135, pupil diameter (using Eye tracking 150), and breathing and heart-related parameters (using physiological measurements 120) to assess proper targeting, potentially even of subnuclei.

Beyond self-reporting of mood, facial expression (using EMG 125, motion detectors 155, or camera 160) could also be used to assay the target and whether a tFUS stimulation or modulation parameter is excitatory or inhibitory.

### Task Based Measurements

System 100 includes mechanisms for providing tasks to subject 101 and evaluating the results. Subject 101 may also undergo tasks with or without explicit instructions. Non-explicit tasks may present stimuli passively without actively telling the subject of the relevance and measure reactions. Explicit tasks beyond subjective reporting may include objective strategic games (risk, memory, etc.) or postural change. Tasks are used after “spatial targeting” methods to refine further targeting for an individual subject. However, the same spatial targeting methods (mainly cameras) can be used to record the adjustments and placements during and after the “functional targeting” is done to record the better positioning and aiming of the transducers. Effects may be more powerfully seen when combined with various tasks. These tasks may be cognitively intense (say memory, attention, detection tasks), passive (detecting eye gaze shifts when subtle, barely noticeable visual stimuli are presented in a small part of visual space or measuring responses to sudden sounds), or simple (postural change tasks like standing up and lying down). Tasks can have power in data: many trials can be run for specific tasks and see trends in time. Some examples of tasks are given below.

Games involving reward, statistical/probability-based games, and gambling, including strategy switching, can be used. The cingulate cortex is often implicated as involved in strategy switching, and beyond choices, other readouts like non-invasive physiological readouts and latencies can be used. In addition, if choices are presented in a left/right manner, spatial bias can also be utilized.

Tasks could include two alternative forced-choice tasks. For example, subject 101 is shown two images and can choose to keep seeing one of the two for a set time or more often. When images are of different emotional valences, one may see a strong, fast reaction to choose the more positive one, while a depressed person may not care as much. Modulation of the amygdala could affect such choices by affecting the encoding of valences and be used as a simple adaptation of predator or shock avoidance models used in animals.

Various brain regions implicated in depression and anxiety, such as the PCC and amygdala, are also involved in memory formation, and their artificial modulation can affect memory. The type of memory affected could involve: General and neutral or personal or emotion-evoking objects, faces, places, etc.

Remembering things presented in order, but several presentations before (e.g., N-back)—these tasks may be used to assess memory formation or attention (for example, it would be considered a task that is very narrowly and externally focused and would be sensitive to PCC modulation)

Spatial navigation (say in a maps app, to navigate a route known to subject 101) or simple spatial memory (which location was object X shown 1 minute ago?). Spatial navigation is often implied in PCC studies.

Details with self-relevance, like the address of your childhood home or your mom's name. These are implied in PCC studies.

Those with emotional valence (angry vs. happy vs neutral faces and scenes). These are often implied in amygdala studies.

Images could be present on the left vs. right side of the brain, and memory biasing due to unilateral tFUS modulation can also be used.

Example tasks involving subject 101 feedback are given below.

For example, instead of asking about subjective mood during amygdala stimulation/modulation, subject 101 is asked to rate faces or video clips on a scale indicating more positive or negative. Such tasks can often be more sensitive to subtle changes than subjective feedback of present mood. In addition, many more trials can be run to provide more robust data and track changes continuously.

Asking how well a short narrative relates to subject 101, which could be modified by PCC modulation.

Detection tasks could involve measurements of the threshold of a sensory stimulus, often visual or auditory, for a person to notice. They would often be lateralized as stimulation/modulation of brain regions that increase or decrease attention or salience often have lateralized effects. These sensory stimuli can be very subtle, like a small moving or suddenly appearing visual patch with low contrast from surround or relatively low amplitude sudden noises in a noisier environment. How strong is the detection threshold contrast (relative to the background)? Is there left/right bias, etc? Subject 101 is asked explicitly to detect changes. tFUS waveforms could be used to improve or decrease performance in detecting a certain contrast or intensity stimulus or shift bias of detection towards a direction.

Note that some of the same tasks can also be done more passively by measuring reflexive gaze shifts (such as for the detection task), changes in pupil size, breathing or heart-related variables (say for the presentation of faces with valences task), galvanic skin responses, or EEG (say lower latency or larger amplitude evoked potentials, for example). For responses to tFUS stimuli/modulation, effects may be indirect (say stimulation/modulation of one location leads to increased vigilance or attention, and hence changes in activity in a primary sensory area) or downstream (for the same sensory input and even activity at the early brain regions, but response downstream changes).

Simple tasks like postural changes can be used to change blood flow, pressure, etc. Peripheral and brain Doppler and other blood/pulse-related measurements can measure these. Patients with anxiety often show abnormal responses to these changes compared to healthy controls. For example, blood flow in healthy patients often normalizes back within a minute of a postural change, while anxious patients do not show this. In addition, measurables like phase differences in blood flow at different locations or relative to heartbeat can also show differences. Furthermore, patients who recover from anxiety with treatment show responses similar to healthy controls, while those who do not get better with treatment show differences.

Tasks may also use visuo-tactile or auditory illusions that induce a sensation of “out-of-body” experiences. Given the PCC's implication in such sensations, the modulation of the PCC will likely affect the strength of such illusions and tasks relying on them. Such tasks could be implemented using VR systems and can be useful and an interesting or enjoyable experience for the subject.

Measurements within a tFUS Session and Across tFUS Sessions

While many of these measurements can be made relatively easily on single subjects and single time points, they may not offer sufficient information for optimal efficacy. Due to high inter-individual differences in brain function and anatomy, even group-level data is relatively hard to apply to a single individual.

In contrast, a number of methods can be applied to elicit higher contrast or more useful information. In particular, intra-subject measurements can be powerful when comparing the effects of before-and-after or intermixed control (aka placebo or sham) and active trials, periods, or sessions. These methods can be used within a session and between sessions (say, across days), allowing more data to be collected and opportunities for optimization.

Consider an example scenario where a specific tFUS waveform and brain region target are thought to cause a 10% change in HRV (heart rate variability). In contrast, placebo treatment is generally known to result in a 5% change based on data obtained from a large group of subjects. However, it may happen that an individual subject, even with true and efficacious stimulation/modulation, only elicits a 5% change in HRV, but this information might not have been available beforehand. Based on this, it may seem that the target has been missed. One might try to change parameters, but the HRV changes may still remain lower. However, conducting a placebo session can reveal that this particular subject yields only a 2.5% change in HRV with placebo sessions. This indicates that the 5% change was a good effect size, and this particular subject does not respond as strongly to this measurement. Therefore, conducting intra-subject, individual comparisons of sham sessions to active sessions can be useful in correcting individual placebo effects as well as response magnitudes.

In addition, such longer-term approaches across multiple sessions can incorporate data from outside sources, such as food journals, sleep logs, personal events, and similar confounding factors, to adjust therapy as needed or account for any unexpected effects.

Subject 101 measurements can help compare the effects of tFUS treatment. Measurement can be done before tFUS treatment to establish a baseline measurement. Measurements can be performed with intermixed control and active trials and periods. These measurements can be used both within and between sessions (say, across days). The measurements can examine physical aspects (such as precision of targeting) and incorporate data from outside sources, such as food journals, sleep logs, personal events, and similar confounding factors, to adjust therapy as needed.

System 100 can measure the effects of tFUS stimulation/modulation in a single treatment session with a single tFUS period, a single treatment session with multiple tFUS periods, or testing across sessions. The measurements may be compared with placebo trials, in which subject 101 thinks they are receiving treatment while they are not. The measurements may also be compared to periods in which tFUS treatment is off.

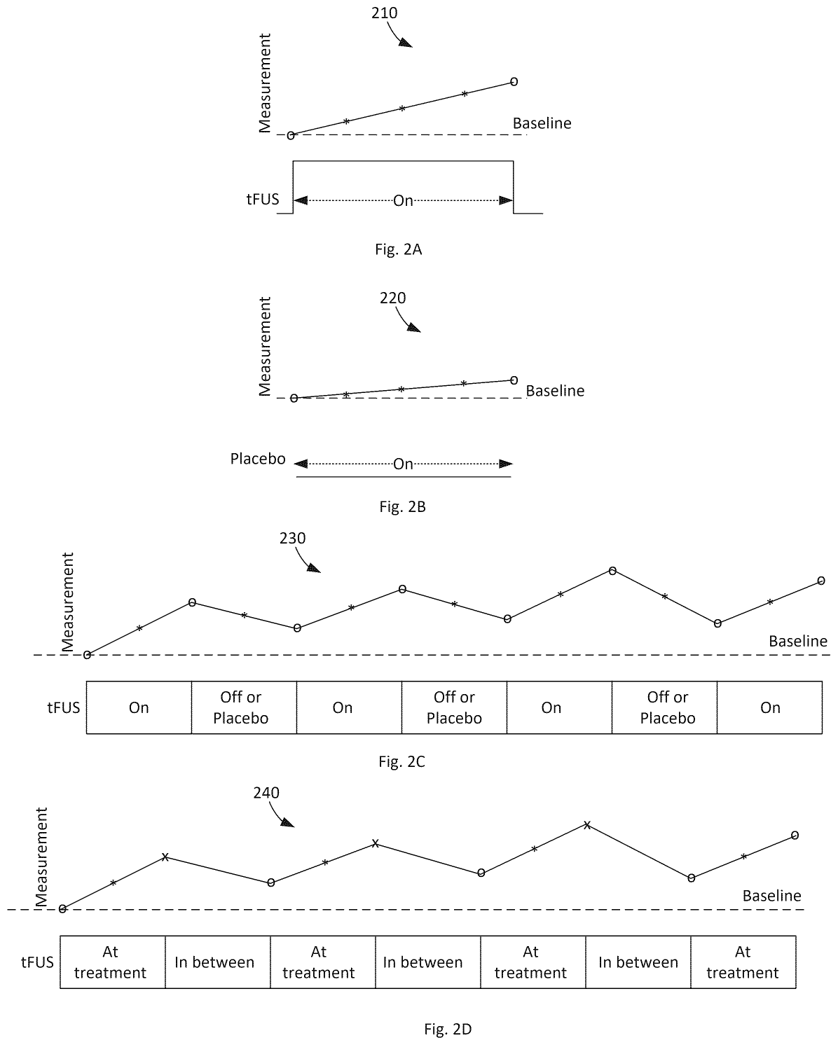

FIG. 2A shows sample measurement 210 within a single tFUS period. Discrete measurements “*” (asterisk) are plotted on a continuous line. The measurements before and after the tFUS period are shown as “o.” FIG. 2B shows a similar measurement 220 but with a placebo. While even a placebo session may show changes, for a useful measurement, the improvement in measurement from the tFUS treatment must be different from those seen in a placebo session. Typically, the changes with real treatment may be larger than those in a placebo session, but that is not always the case. For example, if one is testing something like attentional performance for a person who is undergoing treatment for attentional issues, then the real treatment would optimally show smaller changes (less drop in performance as the session goes on).

FIG. 2C shows sample measurement 230 within a single treatment session consisting of multiple interspersed periods of tFUS and an “off” or “placebo” condition. These may measure changes within Subject 101 in a single session. Such “on-off” treatment may be motivated by other considerations, including safety or efficacy, as well as convenience for the subject such as restroom breaks, breaks from performing a task, or to allow standing up and moving around a bit. The measurements could be done continuously through the entire session (line), at discrete points in between (“*,” asterisk), or at or near the transition time points between the on and “off/placebo” conditions (“0”).

FIG. 2D shows sample measurement 240 across multiple treatment sessions. Effective treatment of mental illnesses and mood disorders should be a long-term result. Tracking measurements across sessions can be done at the beginning (“o”), during (“*,” asterisk), or end (“x” mark) of sessions or a combination of these. These measurements can ensure steady improvement and a long-lasting therapeutic effect.

### Targeting

In addition to adjusting targeting and optimizing waveforms using measurables expected from the target brain region, an approach applicable when the target brain region itself may not offer any immediate readouts but is surrounded by regions that may be used instead for triangulation or waveform optimization is described.

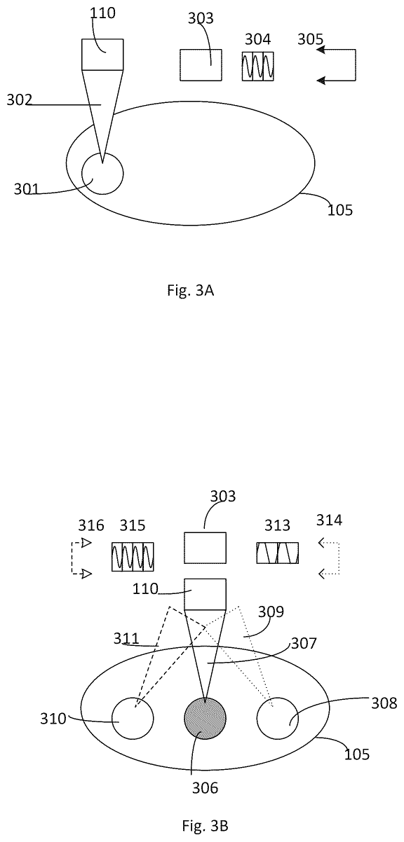

FIGS. 3A and 3B illustrate targeting examples using System 100. FIG. 3A shows an example of direct targeting, while FIG. 3B shows indirect targeting. System 100 can be used even if the desired target brain region has no immediately noticeable, measurable, or quantifiable effects with tFUS stimulation/modulation waveforms. System 100 can generate an individualized targeting map if the target brain region is surrounded by regions that provide feedback and the spatial arrangement of these surrounding regions is known. In FIG. 3A, Transducer array assembly 110 targets anatomical brain region 301 of subject 101 using tFUS waveform 302. Measurement systems 303, which consist of one or more combinations of physiological measurements, physical measurements (eye, movement, etc.), or task performance measurements, measure the effects of tFUS waveform 302. The measurements are quantified. This is shown as waveform 304. System 100 detects changes 305 when target 301 is properly targeted spatially and with an effective modulating tFUS waveform 302.

In FIG. 3B, anatomical target 306 inside subject 101's head 105 does not yield immediately noticeable, measurable, or quantifiable effects with tFUS waveforms 307 from transducer array assembly 110. However, targets 308 and 310 yield measurable changes when stimulated/modulated by tFUS waveforms 309 and 311, respectively. Targets 308 and 310 have known relatively consistent locations relative to target 306. tFUS waveforms 309 and 311 may have differences beyond the targeting, including spatial features, temporal features, pressure amplitudes, and other relevant parameters of the ultrasound field. Measurement systems 303, which consist of one or more combinations of physiological measurements, physical measurements (eye, movement, etc.), or task performance measurements, measure the effects of tFUS waveform 309 on target 308. System 100 quantifies the measurements and is shown as waveform 313 in the figure. System 100 detects changes 314 when target 308 is properly targeted spatially and with an effective modulating tFUS waveform 309. Similarly, for target 310 stimulated/modulated with tFUS waveform 311, System 100 quantifies the measurements as 315 and quantifies the change 316 when target 310 is properly targeted. Measurements 313 and 315 can be different. For example, Measurement 313 occurs in GSR 130 and reported valence of images, while Measurement 315 occurs in heart rate (120) and memory performance. The direction or type of change 314 and 316 may be different. Using targeting to regions 308 and 310, an improved individualized targeting to brain region 306 is achieved. In this example, only two other brain regions are shown. One, two, three, or any number of brain regions that evoke a measurable response can be used. Efficacy for subject 101 may be assumed for what worked in the other regions (308, 310) nearby or perhaps a control region known to be similar in responses.

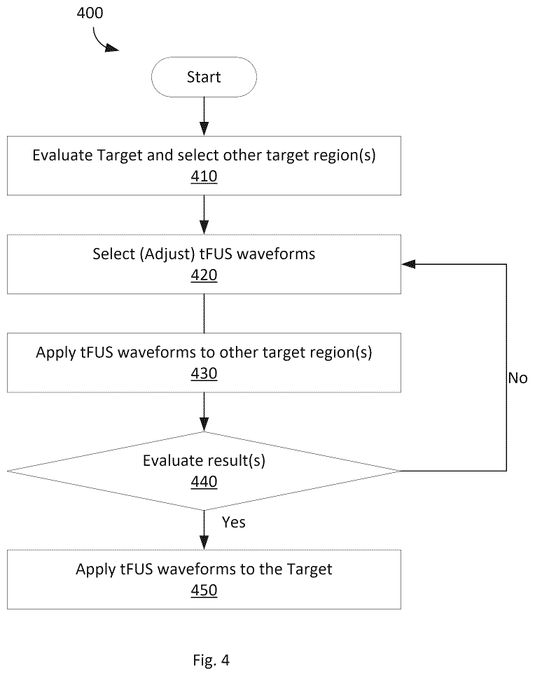

FIG. 4 shows an exemplary method 400 used by System 100 for indirect targeting. Method 400 starts at Operation 410, where the target anatomical region inside subject 101's head 105 is evaluated. The target region inside subject 101's head 105 does not yield immediately noticeable, measurable, or quantifiable effects with tFUS waveforms from tFUS Transducer array assembly 110. One or more nearby target regions that yield measurable changes when stimulated/modulated by tFUS waveforms are selected. The selected nearby target regions that yield measurable changes have known relatively consistent locations relative to the target. For example, referring to FIG. 3B, target 306 is evaluated, and nearby targets regions 308 and 310 are selected. The spatial orientation between target 306 and regions 308 and 310 is known.

Referring to FIG. 4, in Operation 420, effective modulatory tFUS waveforms for the nearby target regions (regions 308 and 310 in the example) are selected. In Operation 430, the selected tFUS waveforms are applied to the nearby selected target regions (regions 308 and 310). In Operation 440, the results from the tFUS waveforms are evaluated. In Operation 440, System 100 measures and quantifies the effects of the stimulation/modulation. System 100 can measure the effects of the tFUS waveforms using direct neural activity measurement 114, physiological measurements, EMG 125, EDA/GSR 130, microneedles/sensors 135, eye monitors 140, motion detectors 155, camera 160, subject 101 or clinician reporting. Measurements can be made during the stimulation/modulation or after the tFUS stimulation/modulation session and can be task-based.

A clinician uses these results to determine if the nearby selected target regions are targeted accurately. If it is determined that selected regions are targeted accurately, the following operation is Operation 450; otherwise, the following operation is either Operation 410 or Operation 420. The clinician determines if the tFUS waveform parameters must be adjusted for correct targeting (Operation 410). If needed, the clinician selects one or more different targets (i.e., Method 400 proceeds to Operation 410). Though Operation 430 and 440 are depicted as a single sequential process in the figure, multiple processes could be performed. For example, in Operation 430, the first selected target region (e.g., 308) is stimulated/modulated, and the results are evaluated in Operation 440. Operations 430 and 440 are repeated for the other selected target regions (e.g., 310). In Operation 450, required adjustments to tFUS waveforms and Transducer array assembly 110 position are made, and the target region is stimulated/modulated.

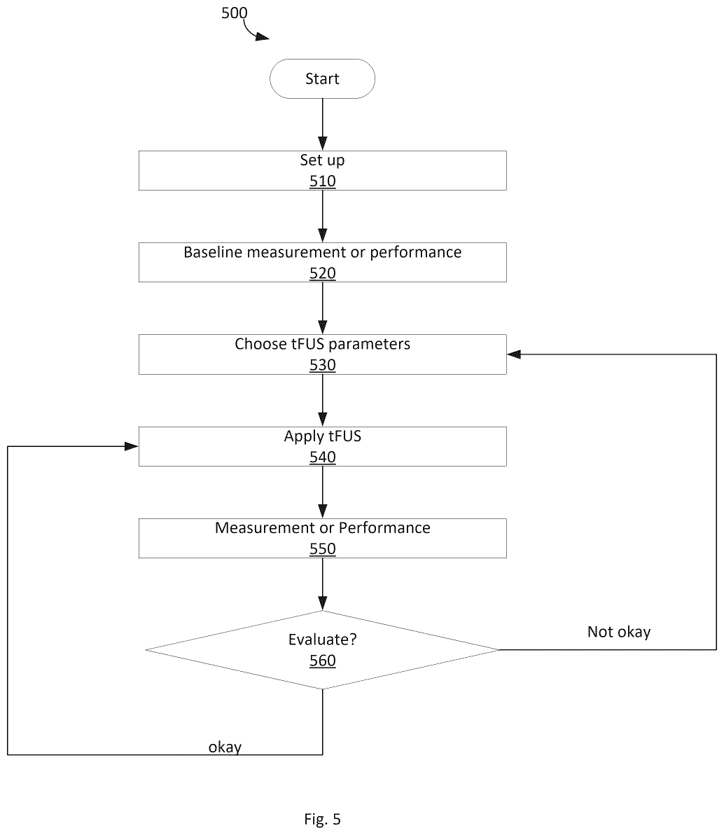

FIG. 5 shows an exemplary method 500 performed using System 100 to evaluate and improve the efficacy of tFUS stimulation/modulation. Method 500 can improve tFUS stimulation/modulation efficacy by adjusting tFUS stimulation/modulation parameters. Method 500 performs a baseline measurement on subject 101 and compares the measurement during a tFUS stimulation/modulation to determine the stimulation/modulation efficacy.

Method 500 starts at Operation 510, where System 100 is set up to measure Subject 101's parameters or performance. Measurements can be performed using direct neural activity measurement 115, physiological measurements 120, EMG 125, EDA/GSR 130, microneedles/sensors 135, eye monitors 140, motion detectors 155, camera 160, or subject 101 or clinician reporting. System 100 can measure subject 101's performance as part of non-explicit or explicit tasks. Transducer array assembly 110 is affixed to Subject 101's head 105. Guidance or spatial targeting is performed for the initial targeting of the required anatomical region.

In Operation 520, subject 101's baseline measurement or performance is measured. This could be a passive measurement of a physiological parameter—e.g., breathing, heart rate, eye movement, facial movement, blood pressure, stress measurement, etc. The measurement could be based on a non-explicit task, where subject 101 is presented with stimuli with no explicit instructions. The measurement or performance measurement can be based on a task with explicit instructions such as a change in posture, memory-based, etc. Measurements are quantified so that the effects of the tFUS stimulation/modulation can be evaluated.

In Operation 530, a clinician chooses the tFUS parameters. In Operation 540, Transducer array assembly 101 is used to apply a tFUS stimulation/modulation waveform on subject 101. In Operation 550, Subject 101's measurement or performance is measured and quantified. The parameters measured in this operation are those measured in Operation 520. System 100 measures the effects of tFUS stimulation/modulation in a single treatment session with a single tFUS period (FIG. 2A), a single treatment session with multiple tFUS periods (FIG. 2C), or across sessions (FIG. 2D).

In Operation 560, the measurements (or performance) from Operation 550 are evaluated against the baseline measurements (or performance, from Operation 520). If the results look acceptable, the following operation is Operation 540, and the tFUS parameters are saved. The saved parameters can be reused for subsequent sessions and help save time and money by avoiding the measurements (baseline and during stimulation/modulation). The cost and system complexity required for subsequent sessions is reduced by avoiding any or some of the measurement systems. Subject 101 can continue the stimulation/modulation session or use the saved parameters for subsequent sessions. If the results are not acceptable, a clinician adjusts the tFUS parameters. Subject 101 can continue the stimulation/modulation session with the adjusted parameters or use them for subsequent sessions.

Individual vs. Group Tracking

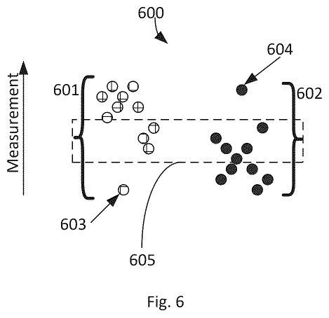

Group measurements can help identify useful modalities of measurements and the expected direction and magnitude of changes with therapy. FIG. 6 shows an exemplary measurement 600 performed using System 100, illustrating individual versus group measurement. FIG. 6 shows data points for healthy controls 601 (pattern) and data points for a patient population 602 (dark-shaded). Comparisons of the group-mean will yield apparent differences, and these can be used to inform the utility of a measurement as a correlate or readout of the indication (say anxiety for patient population). However, at the individual level, there will be large variability. A healthy individual may be an extreme outlier 603, showing measurements similar to the patient population. Similarly, a patient could be an extreme outlier 604, showing measurements more similar to healthy controls. In addition, there will often be an intermediate band 605 where the healthy and patient data points overlap.

Thus, while this measurement shows promise, simple group or individual-to-group comparisons alone may not always be useful or accurately classify people. However, just because the measurement method and modality yielding outlier 604 shows that it is within a healthy range doesn't mean it isn't useful for this individual as a measurement method. If the measurement changes in response to tFUS stimulation/modulation in the correct way, it can possibly still be used for this individual. Thus, these measurements can be analyzed in individuals across conditions and time to assess therapy efficacy even if the group data does not easily apply.

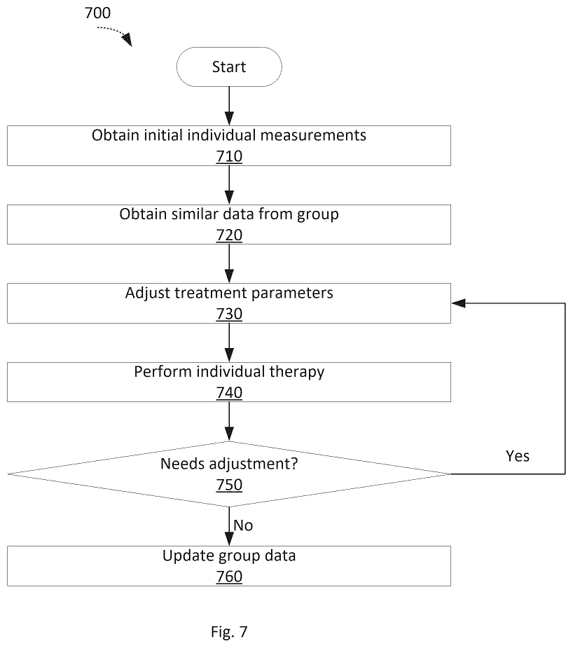

FIG. 7 is an exemplary method 700 performed using System 100 to adjust individual and group measurements. Group measurements can be used as an initial starting point for an individual treatment. The treatment or tFUS parameters can be adjusted based on the individual's results. Group data can be updated to reflect individual results. Method 700 starts at Operation 710, where initial measurements are performed on an individual or subject 101. Before the initial measurement, subject 101 is set up within System 100. Any required spatial measurements are performed to help customize the group results to the individual or subject 101.

At Operation 720, the initial results obtained from Operation 710 are evaluated, and similar data from the group data is selected after weighing different measurements, prioritization, targets, waveforms, etc.

In Operation 730, a clinician optionally adjusts the treatment parameters based on the initial measurements performed in Operation 710.

At Operation 740, treatment is performed on subject 101. System 100 measures the effects of the treatment.

At Operation 750, the results are evaluated to see if any adjustments to treatment parameters are required. If adjustments are required, the following Operation is Operation 730. Otherwise, the following operation is Operation 760.

At Operation 760, the group data is updated with subject 101's results. The results help other individuals with similar symptoms or seeking similar treatment.

Individualized Functional Targeting for at-Home Use

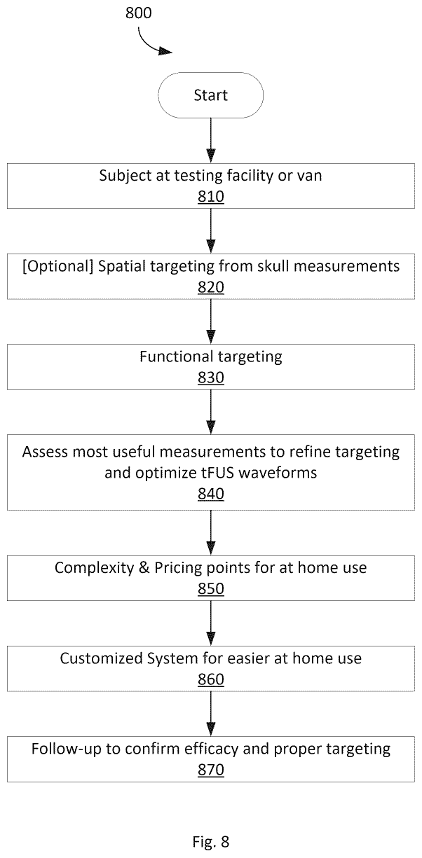

It is not practical or economical to have all of the different measurement systems in System 100 for use at an individual level, especially in a non-clinical setting like home. FIG. 8 shows an exemplary Method 800 that enables functional targeting of a subject for at-home use. Method 800 starts at Operation 810, where subject 101 (or individual) is in a clinic or testing facility. The facility can be mobile, similar to a van well-equipped with the different testing or measuring systems of System 100.

At Operation 820, spatial targeting is optionally performed on Subject 101. The spatial targeting could use skull measurements from MRI, CT, camera, or LIDAR. As the individual responses vary, just using spatial targeting might not be sufficient.

At Operation 830, the targeting is fine-tuned using functional targeting, where the downstream effects of the tFUS waveforms are measured and quantified. As described previously, the downstream effects include physiological measurements, facial expressions, eye movements, stress measurements, task-based performance, etc.

At Operation 840, the results from Operation 830 are assessed, and the most useful measurements pertaining to subject 101 are selected to refine targeting and optimizing tFUS waveforms. The downstream effects are highly individualistic, where for the same treatment, one can show a distinct measurement in blood pressure, and a different person can show a distinct measurement in a different aspect, for example, performance in a task, etc. The measurement modalities selected for subject 101 are also assessed for complexity and price, where the least complex and cheapest are prioritized.

At Operation 850, subject 101 is presented with treatment plans, the system's complexity, and price points. Based on their preferences, subject 101 selects treatment plans and system options.

At Operation 860, System 100 is customized so that Subject 101 can use it at home and make it easier. Customization could include removing unwanted components (e.g., measurement systems that Subject 101 does not show a distinct measurement to downstream effects), streamlining the flow to suit the subject, etc. It can also include printing custom 3D attachments so Subject 101 can easily attach the system to their head or attach measurement systems to the system.

At Operation 870, Subject 101 periodically or on a need-basis follows up with a clinician or testing facility to review results. Subject 101 can confirm the system's proper targeting and efficacy.
1. In some embodiments, a transcranial-focused ultrasound system (tFUS) delivery and tracking system comprises a transducer array assembly configured to deliver a tFUS waveform to a subject, a direct neural activity measurement system measuring neural activity in response to the tFUS waveform, a physiological measurement system measuring a physiological response to the tFUS waveform, an electromyography (EMG) system, and a user interface device displaying data from at least one of the direct neural activity measurement system, the physiological measurement system, or the EMG system.
2. The tFUS delivery and tracking system of clause 1, wherein the physiological measurement system measures at least one of circulation, respiration, blood pressure, blood flow, blood oxygen saturation, blood or end-tidal CO2, heartbeat strength, heart rate and rate variability, nasal airflow parameters, oral or nasal thermal measurement, temperature, or blood sugar of the subject.
3. The tFUS delivery and tracking system of clauses 1 or 2, further comprising an anatomical targeting system for targeting a position on a skull or brain of the subject.
4. The tFUS delivery and tracking system of any of clauses 1-3, wherein the transducer array assembly delivers a tFUS waveform comprising at least one of an excitatory, an inhibitory, or a modulatory waveform that modulates the magnitude of induced activity in the brain region in response to stimuli.
5. The tFUS delivery and tracking system of any of clauses 1-4, further comprising a stress and sympathetic measurement to measure stress and sympathetic responses to the tFUS waveform.
6. The tFUS delivery and tracking system of any of clauses 1-5, wherein the transducer array assembly indirectly targets the target region in response to the tFUS waveform yielding no measurable or quantifiable effect on the target region.
7. The tFUS delivery and tracking system of any of clauses 1-6, wherein the direct neural activity measurement system comprises at least one of EEG, MEG, fMRI, or fNIRS.
8. In some embodiments, a method comprises determining that a first transcranial focused ultrasound (tFUS) waveform directly targeted at a first target region does not yield measurable effects in response to the waveform, selecting at least one alternative target region in response to the determination that the first tFUS waveform directly targeted at the target region does not produce measurable effects, determining that effects of applying at least a second tFUS waveform to at least one alternative target region are measurable, and applying tFUS waveforms to the first target region in response to determining that at least a second tFUS waveform applied to at least one alternative target region is measurable.
9. The method of clause 8, wherein determining the effects of applying at least a second tFUS waveform to at least one alternative target region is based on data from a direct neural activity measurement system measuring neural activity in response to the tFUS waveform.
10. The method of clauses 8 or 9, wherein the direct neural activity measurement system comprises at least one of EEG, MEG, fMRI, or fNIRS.
11. The method of any of clauses 8-10, wherein determining the effects of applying at least a second tFUS waveform to at least one alternative target region is based on data from an electromyography (EMG) system.
12. The method of any of clauses 8-11, wherein determining the effects of applying at least a second tFUS waveform to at least one alternative target region is based on data from a stress and sympathetic measurement to measure stress and sympathetic responses to the tFUS waveform.
13. The method of any of clauses 8-12, wherein determining the effects of applying at least a second tFUS waveform to at least one alternative target region is based on data from a physiological measurement system measuring a physiological response to the tFUS waveform.
14. The method of any of clauses 8-13, wherein the physiological measurement system measures at least one of circulation, respiration, blood pressure, blood flow, blood oxygen saturation, blood or end-tidal CO2, heartbeat strength, heart rate and rate variability, nasal airflow parameters, oral or nasal thermal measurement, temperature, or blood sugar of the subject.
15. The method of any of clauses 8-14, wherein the first tFUS waveform or the second tFUS waveform comprises at least one of an excitatory, an inhibitory, or a modulatory waveform that modulates the magnitude of induced activity in the brain region in response to stimuli.
16. The method of any of clauses 8-15, wherein the parameters of the first tFUS waveform applied to the first target region are modified based upon the measurable effects of applying at least a second tFUS waveform to the at least one alternate target region and its relative anatomical location to the first target region.
17. In some embodiments, a method comprises measuring a subject baseline using at least one of a direct neural activity measurement system, a physiological measurement system, or an electromyography (EMG) system, selecting at least one parameter for a transducer array assembly configured to deliver a tFUS waveform to the subject based on the subject baseline, and applying the tFUS waveform to the subject based on at least one parameter.
18. The method of clause 17, further comprising detecting a subject response to the tFUS waveform, and in response to determining that the subject response fails to meet a threshold, selecting at least one different parameter for the transducer array assembly, a different adjustment or correction to targeting, or a different tFUS waveform to the subject.
19. The method of clauses 17 or 18, wherein the direct neural activity measurement system comprises an EEG, MEG, fMRI, or fNIRS.
20. The method of any of clauses 17-19, wherein selecting at least one parameter for a transducer array assembly is based on data from an electromyography (EMG) system.
21. The method of any of clauses 17-20, wherein selecting at least one parameter for a transducer array assembly is based on data from a stress and sympathetic measurement to measure stress and sympathetic responses to the tFUS waveform.
22. The method of any of clauses 17-21, wherein selecting at least one parameter for a transducer array assembly is based on data from a physiological measurement system measuring a physiological response to the tFUS waveform.
23. The method of any of clauses 17-22, wherein the physiological measurement system measures at least one of circulation, respiration, blood pressure, blood flow, blood oxygen saturation, blood or end-tidal CO2, heartbeat strength, heart rate and rate variability, nasal airflow parameters, oral or nasal thermal measurement, temperature, or blood sugar of the subject.
24. The method of any of clauses 17-23, wherein the tFUS waveform comprises at least one of an excitatory, an inhibitory, or a modulatory waveform that modulates the magnitude of induced activity in the brain region in response to stimuli.
25. In some embodiments, a method comprises determining initial measurements of a subject using at least one of a direct neural activity measurement system, a physiological measurement system, or an electromyography (EMG) system, obtaining group data associated with a group of subjects based on direct neural activity measurement system, a physiological measurement system or an electromyography (EMG) system, modifying at least one parameter for a transducer array assembly configured to deliver a tFUS waveform to the subject based on the group data, applying the tFUS waveform to the subject based on at least one parameter, obtaining a response of the subject to the tFUS waveform, in response to determining that the subject response fails to meet a threshold, selecting at least one different parameter for the transducer array assembly, and updating group data associated with a group of subjects based on the response of the subject.
26. The method of clause 25, wherein the direct neural activity measurement system comprises an EEG, MEG, fMRI, or fNIRS.
27. The method of clauses 25 or 26, wherein determining the effects of applying the tFUS waveform is based on data from an electromyography (EMG) system.
28. The method of any of clauses 25-27, wherein determining the effects of applying the tFUS waveform is based on data from a stress and sympathetic measurement to measure stress and sympathetic responses to the tFUS waveform.
29. The method of any of clauses 25-28, wherein determining the effects of applying the tFUS waveform is based on data from a physiological measurement system measuring a physiological response to the tFUS waveform.
30. The method of any of clauses 25-29, wherein the physiological measurement system measures at least one of circulation, respiration, blood pressure, blood flow, blood oxygen saturation, blood or end-tidal CO2, heartbeat strength, heart rate and rate variability, nasal airflow parameters, oral or nasal thermal measurement, temperature, or blood sugar of the subject.
31. The method of any of clauses 25-30, wherein the tFUS waveform comprises at least one of an excitatory, an inhibitory, or a modulatory waveform that modulates the magnitude of induced activity in the brain region in response to stimuli.
32. In some embodiments, a method comprises targeting a transcranial focused ultrasound (tFUS) waveform at a target region, identifying subject responses or measurement to the tFUS waveform indicating effectiveness of the tFUS waveform, determining which measurement systems are most useful for evaluating the effectiveness of the tFUS waveform, further selecting the measurement system based on price and complexity, and customizing the tFUS system by including only the selected measurement system.
33. The method of clause 32, wherein the tFUS waveform comprises at least one of an excitatory, an inhibitory, or a modulatory waveform that modulates the magnitude of induced activity in the brain region in response to stimuli.

Any and all combinations of any of the claim elements recited in any of the claims and/or any elements described in this application, in any fashion, fall within the contemplated scope of the present invention and protection.

The descriptions of the various embodiments have been presented for purposes of illustration but are not intended to be exhaustive or limited to the embodiments disclosed. Many modifications and variations will be apparent to those of ordinary skill in the art without departing from the scope and spirit of the described embodiments.

Aspects of the present embodiments can be embodied as a system, method or computer program product. Accordingly, aspects of the present disclosure can take the form of an entirely hardware embodiment, an entirely software embodiment (including firmware, resident software, micro-code, etc.) or an embodiment combining software and hardware aspects that can all generally be referred to herein as a “module,” a “system,” or a “computer.” In addition, any hardware and/or software technique, process, function, component, engine, module, or system described in the present disclosure can be implemented as a circuit or set of circuits. Furthermore, aspects of the present disclosure can take the form of a computer program product embodied in one or more computer readable medium having computer readable program code embodied thereon.

Any combination of one or more computer readable medium can be utilized. The computer readable medium can be a computer readable signal medium or a computer readable storage medium. A computer readable storage medium can be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium can be any tangible medium that can contain or store a program for use by or in connection with an instruction execution system, apparatus, or device.

Aspects of the present disclosure are described above with reference to flowchart illustrations and/or block diagrams of methods, apparatus (systems) and computer program products according to embodiments of the disclosure. It will be understood that each block of the flowchart illustrations and/or block diagrams, and combinations of blocks in the flowchart illustrations and/or block diagrams, can be implemented by computer program instructions. These computer program instructions can be provided to a processor of a general-purpose computer, special purpose computer, or other programmable data processing apparatus to produce a machine. The instructions, when executed via the processor of the computer or other programmable data processing apparatus, enable the implementation of the functions/acts specified in the flowchart and/or block diagram block or blocks. Such processors can be, without limitation, general purpose processors, special-purpose processors, application-specific processors, or field-programmable gate arrays.

The flowchart and block diagrams in the figures illustrate the architecture, functionality, and operation of possible implementations of systems, methods and computer program products according to various embodiments of the present disclosure. In this regard, each block in the flowchart or block diagrams can represent a module, segment, or portion of code, which comprises one or more executable instructions for implementing the specified logical function. It should also be noted that, in some alternative implementations, the functions noted in the block can occur out of the order noted in the figures. For example, two blocks shown in succession may, in fact, be executed substantially concurrently, or the blocks can sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

While the preceding is directed to embodiments of the present disclosure, other and further embodiments of the disclosure can be devised without departing from the basic scope thereof, and the scope thereof is determined by the claims that follow.

What is claimed is:

1. A transcranial-focused ultrasound system (tFUS) delivery and tracking system comprising:
a transducer array assembly configured to deliver a tFUS waveform to a subject;
a direct neural activity measurement system measuring neural activity in response to the tFUS waveform;
a physiological measurement system measuring a physiological response to the tFUS waveform;
an electromyography (EMG) system; and
a user interface device displaying data from at least one of the direct neural activity measurement system, the physiological measurement system, or the EMG system.

2. The tFUS delivery and tracking system of claim 1, wherein the physiological measurement system measures at least one of circulation, respiration, blood pressure, blood flow, blood oxygen saturation, blood or end-tidal CO2, heartbeat strength, heart rate and rate variability, nasal airflow parameters, oral or nasal thermal measurement, temperature, or blood sugar of the subject.

3. The tFUS delivery and tracking system of claim 1, further comprising an anatomical targeting system for targeting a position on a skull or brain of the subject.

4. The tFUS delivery and tracking system of claim 1, wherein the transducer array assembly delivers a tFUS waveform comprising at least one of an excitatory, an inhibitory, or a modulatory waveform that modulates the magnitude of induced activity in the brain region in response to stimuli.

5. The tFUS delivery and tracking system of claim 1, further comprising a stress and sympathetic measurement to measure stress and sympathetic responses to the tFUS waveform.

6. The tFUS delivery and tracking system of claim 1, wherein the transducer array assembly indirectly targets the target region in response to the tFUS waveform yielding no measurable or quantifiable effect on the target region.

7. The tFUS delivery and tracking system of claim 1, wherein the direct neural activity measurement system comprises at least one of EEG, MEG, fMRI, or fNIRS.

8. A method comprising:
determining that a first transcranial focused ultrasound (tFUS) waveform directly targeted at a first target region does not yield measurable effects in response to the waveform;
selecting at least one alternative target region in response to the determination that the first tFUS waveform directly targeted at the target region does not produce measurable effects;
determining that effects of applying at least a second tFUS waveform to at least one alternative target region are measurable; and
applying tFUS waveforms to the first target region in response to determining that at least a second tFUS waveform applied to at least one alternative target region is measurable.

9. The method of claim 8, wherein determining the effects of applying at least a second tFUS waveform to at least one alternative target region is based on data from a direct neural activity measurement system measuring neural activity in response to the tFUS waveform.

10. The method of claim 9, wherein the direct neural activity measurement system comprises at least one of EEG, MEG, fMRI, or fNIRS.

11. The method of claim 8, wherein determining the effects of applying at least a second tFUS waveform to at least one alternative target region is based on data from an electromyography (EMG) system.

12. The method of claim 8, wherein determining the effects of applying at least a second tFUS waveform to at least one alternative target region is based on data from a stress and sympathetic measurement to measure stress and sympathetic responses to the tFUS waveform.

13. The method of claim 8, wherein determining the effects of applying at least a second tFUS waveform to at least one alternative target region is based on data from a physiological measurement system measuring a physiological response to the tFUS waveform.

14. The method of claim 13, wherein the physiological measurement system measures at least one of circulation, respiration, blood pressure, blood flow, blood oxygen saturation, blood or end-tidal CO2, heartbeat strength, heart rate and rate variability, nasal airflow parameters, oral or nasal thermal measurement, temperature, or blood sugar of the subject.

15. The method of claim 8, wherein the first tFUS waveform or the second tFUS waveform comprises at least one of an excitatory, an inhibitory, or a modulatory waveform that modulates the magnitude of induced activity in the brain region in response to stimuli.

16. The method of claim 8, wherein the parameters of the first tFUS waveform applied to the first target region are modified based upon the measurable effects of applying at least a second tFUS waveform to the at least one alternate target region and its relative anatomical location to the first target region.

17. A method comprising:
measuring a subject baseline using at least one of a direct neural activity measurement system, a physiological measurement system, or an electromyography (EMG) system;
selecting at least one parameter for a transducer array assembly configured to deliver a tFUS waveform to the subject based on the subject baseline; and
applying the tFUS waveform to the subject based on at least one parameter.

18. The method of claim 17, further comprising:
detecting a subject response to the tFUS waveform; and
in response to determining that the subject response fails to meet a threshold, selecting at least one different parameter for the transducer array assembly, a different adjustment or correction to targeting, or a different tFUS waveform to the subject.

19. The method of claim 17, wherein the direct neural activity measurement system comprises an EEG, MEG, fMRI, or fNIRS.

20. The method of claim 17, wherein selecting at least one parameter for a transducer array assembly is based on data from an electromyography (EMG) system.

21. The method of claim 17, wherein selecting at least one parameter for a transducer array assembly is based on data from a stress and sympathetic measurement to measure stress and sympathetic responses to the tFUS waveform.

22. The method of claim 17, wherein selecting at least one parameter for a transducer array assembly is based on data from a physiological measurement system measuring a physiological response to the tFUS waveform.

23. The method of claim 22, wherein the physiological measurement system measures at least one of circulation, respiration, blood pressure, blood flow, blood oxygen saturation, blood or end-tidal CO2, heartbeat strength, heart rate and rate variability, nasal airflow parameters, oral or nasal thermal measurement, temperature, or blood sugar of the subject.

24. The method of claim 17, wherein the tFUS waveform comprises at least one of an excitatory, an inhibitory, or a modulatory waveform that modulates the magnitude of induced activity in the brain region in response to stimuli.

25. A method comprising:
determining initial measurements of a subject using at least one of a direct neural activity measurement system, a physiological measurement system, or an electromyography (EMG) system;
obtaining group data associated with a group of subjects based on direct neural activity measurement system, a physiological measurement system or an electromyography (EMG) system;
modifying at least one parameter for a transducer array assembly configured to deliver a tFUS waveform to the subject based on the group data;
applying the tFUS waveform to the subject based on at least one parameter;
obtaining a response of the subject to the tFUS waveform;
in response to determining that the subject response fails to meet a threshold, selecting at least one different parameter for the transducer array assembly; and
updating group data associated with a group of subjects based on the response of the subject.

26. The method of claim 25, wherein the direct neural activity measurement system comprises an EEG, MEG, fMRI, or fNIRS.

27. The method of claim 25, wherein determining the effects of applying the tFUS waveform is based on data from an electromyography (EMG) system.

28. The method of claim 25, wherein determining the effects of applying the tFUS waveform is based on data from a stress and sympathetic measurement to measure stress and sympathetic responses to the tFUS waveform.

29. The method of claim 25, wherein determining the effects of applying the tFUS waveform is based on data from a physiological measurement system measuring a physiological response to the tFUS waveform.

30. The method of claim 29, wherein the physiological measurement system measures at least one of circulation, respiration, blood pressure, blood flow, blood oxygen saturation, blood or end-tidal CO2, heartbeat strength, heart rate and rate variability, nasal airflow parameters, oral or nasal thermal measurement, temperature, or blood sugar of the subject.

31. The method of claim 25, wherein the tFUS waveform comprises at least one of an excitatory, an inhibitory, or a modulatory waveform that modulates the magnitude of induced activity in the brain region in response to stimuli.

32. A method comprising:
targeting a transcranial focused ultrasound (tFUS) waveform at a target region;
identifying subject responses or measurement to the tFUS waveform indicating effectiveness of the tFUS waveform;
determining which measurement systems are most useful for evaluating the effectiveness of the tFUS waveform;
further selecting the measurement system based on price and complexity; and
customizing the tFUS system by including only the selected measurement system.

33. The method of claim 32, wherein the tFUS waveform comprises at least one of an excitatory, an inhibitory, or a modulatory waveform that modulates the magnitude of induced activity in the brain region in response to stimuli.
